(19) United States
(12) Patent Application Publication
(10) Pub. No.: US 2017/0165123 A1
(43) Pub. Date: Jun. 15, 2017

(54) Absorbent item having indicators for the differential detection of urine and faeces

(71) Applicant: Roberto José GOGIN FENANDEZ, Lima (PE)

(72) Inventor: Roberto Jose Gogin, Lima (PE)

(21) Appl. No.: 15/315,525

(22) PCT Filed: Jun. 20, 2014
(86) PCT No.: PCT/PE2014/000008

§ 371 Date: Dec. 8, 2016

(51) Int. Cl. A61F 13/42; A61L 15/24; A61L 15/56

(52) U.S. Cl. CPC A61F 13/42; A61L 15/56; A61L 15/24

(57) ABSTRACT

The invention relates to a disposable absorbent item comprising an absorbent core, lateral leg elastics, a front band, a back elastic, and adhesive tape at the ends of the back elastic, characterised in that a pair of tapes are provided on the external part of the absorbent core, wherein one tape presents an indicator for detecting urine with a pH between 3 and 5, and the other tape presents said indicator in an acid solution for detecting faeces, thereby permitting a differential identification of urine and faeces.

Absorbent Article With indicators differential detection between urine and feces. And the inclusion of these indicators in the production process of the absorbent articles.

### TECHNICAL FIELD OF THE INVENTION

This invention relates to an absorbent article with the ability to detect the secretion of urine and feces secretion differentially by externally visible indicators.

Also proposing to include these indicators to existing production processes of the absorbent articles.

### BACKGROUND OF THE INVENTION

While there are patents urine detection by different chemical reactions and externally exposed in the disposable diaper, we found no difference that allows secretions, validating whether it is urine or feces.

Patent WO 2007073139 A1 diagnosis diaper including a plurality of reagents sensitive indicators of urine and excrement, attached to the inner cover of the absorbent article, serves to monitor the urine and feces of patient for substances not found in common situations in urine and feces, he watched the colorations that give different reagents indicators absorbent article establishes whether a substance present abnormally.

On the other hand, although the versions found in identifying moisture absorbent article market, we see that these versions are not sold in all markets and regularly is more expensive lines.

Patent absorbent article for incontinence EN 1065290U, refers to an absorbent article incorporating a moisture indicator between an absorbent core fixed to an impermeable sheet, such that upon contact with urine, change color, or disappear, giving information to the caregiver presence of urine and the saturation level of the absorber and its need to change, reducing or eliminating the risk of urine leakage occur.

WO 99/16401 discloses an absorbent article having a wetness indicator on the inside of the material of the backing sheet. The material of the backing sheet is a laminate of a fibrous nonwoven material and a polymer sheet. A plurality of translucent windows are arranged in said backsheet through which is visible the humidity indicator.

The object of the present invention to present a choice of indicators in disposable absorbent articles to differentiate secretion of urine and feces external visible form the absorbent article, and this option is readily applicable to existing production processes.

### SUMMARY

The invention relates to a disposable absorbent item comprising an absorbent core, lateral leg elastics, a front band, a back elastic, and adhesive tape at the ends of the back elastic, characterised in that a pair of tapes are provided on the external part of the absorbent core, wherein one tape presents an indicator for detecting urine with a pH between 3 and 5, and the other tape presents said indicator in an acid solution for detecting faeces, thereby permitting a differential identification of urine and faeces.

### DESCRIPTION OF THE INVENTION

The present invention includes in a first aspect an absorbent disposable article detection indicators urine and feces differently and, in second aspect, a process for manufacturing such absorbent article.

According to a first aspect of this invention there is provided a disposable absorbent article formed by a front waist region, rear waist region, a crotch region, the front and rear waist regions together along side edge portions being transversely opposite extending in vertical direction in the regions of front and rear waist in order to form a waist opening and a pair of leg openings, and a pair of fastening tapes on the back waist region. The disposable diaper further comprises two tapes of absorbent paper or filter paper, placed along the diaper in the vertical direction from the front waist to back waist. Here we describe the composition of the tapes:

### Tape No 1

Filter paper or absorbent paper impregnated with bromophenol blue (C19H10O55Br4), prepared at 0.1% in ethyl alcohol 20%, achieving an indicator that detects pH between 3 and 5.

This tape indicates the presence of both urine and feces, with a violet-purple blue. Must have a thickness of 0.5 mm and a width of 6.0 mm, the length will depend on the size of the disposable absorbent article.

### Tape No 2

Absorbent paper or filter paper impregnated acetic acid, sodium acetate salt and bromophenol blue (C19H10O55Br4), prepared 0.1% in ethyl alcohol 20%, in the order of application. We rely on the concept known as “protein error indicators”, a phenomenon that is characterized by the color change point of some indicators of pH is different in the presence of proteins.

If we consider the difference between the amount of protein present in feces vs those present in the urine, this film shows the exclusive presence of feces, with a violet-purple blue. Must have a thickness of 0.5 mm and a width of 6.0 mm, the length will depend on the size of the absorbent article.

Considering the diaper inclusion of these two tapes would have 3 options in using the absorbent article:

1. The absorbent article conventional: there was secretion of urine, or feces. FIG. 1
2. The absorbent article with the tape (6) blue violet-purple color, the presence of urine or feces. FIG. 2
3. The absorbent article with the tape (6) blue violet-purple color, the presence of urine or feces and ribbon (7) in blue purple violet, the presence of stool. FIG. 3

In short, when Lee secretion occurs, we lit the two tapes, whereas if urine secretion occurs, we turned on only one of the tapes.

According to a second aspect of the invention there is provided a process to include the indicators described in the production process of the disposable article.

Considering the phases of production:
First stage—Defibration: The process begins at the mill, where a sheet of pulp enters to be converted into fibers. These fibers are deposited on a forming drum by vacuum. While the fibers are deposited on the former, they are mixed with an absorbent in the vacuum chamber of the drum. The drum form an absorbent package.
Second phase—Formation of core: Once the package (mixture of absorbent and cellulose) acquires the desired shape, is transported by a paper web, which may be on top or around the package. The package is compressed through trouser roller, and is then cut into individual parts
Third phase—Assembly: A strip of polyethylene film or a waterproof fed into the bottom of the package. The headband was previously added to the film by using an applicator. For all these materials can be joined, hot melt adhesive was called hot mixture. The elastic are added in this section and attached with adhesive.
Fourth stage—Application tape: The next step in the process is the addition of the side straps, which are placed with a cutter and applicator device. They can be adhesive tapes or ribbons Sailboat (MR) type.
Fifth phase—Bending and cutting: diapers strip continues toward a rotary knife, which is responsible d how the area of the legs and removes the excess. The diaper continues to bending section (longitudinal and transverse) and cut where finished diapers are obtained.
Sixth phase—counting and packaging: After passing a counter and automatic stacker and pocketing in its final packaging.

We review process modification for efficient inclusion of the indicator tapes, as follows:
Phase Two, when compressing the package need to include in the two rows forming mold (8) by way of windows that allow the reduced step stool and urine, the reaction can be generated conspicuously and in contact the test strips. The grooves must have an entry between 2.0 and 3.0 mm and an output of 1.0 mm, as shown in FIG. 4.
Third Phase on the strip including polyethylene and test strips are described above, in the same position of the absorbent article which are grooves (8), the tapes will allow detection of fluids also serve as extra support to the impermeable polyethylene strip, in order to avoid any leakage.

The other steps of the phases described, and the other phases of due process, should be maintained as described above.

Notably, both the formation of the grooves as the inclusion of test strips not significantly affect the standard cost of the absorbent article (less than 5%), taking into account a cost of production benchmark of USD 0.02 per unit.

### BRIEF DESCRIPTION OF THE FIGURES

FIG. 1: shows a perspective view partly in section of an absorbent article open, showing the different parts of the absorbent article and absorbent core (1), lateral spring legs (2), front band (3), spring back (4) and tape (5).

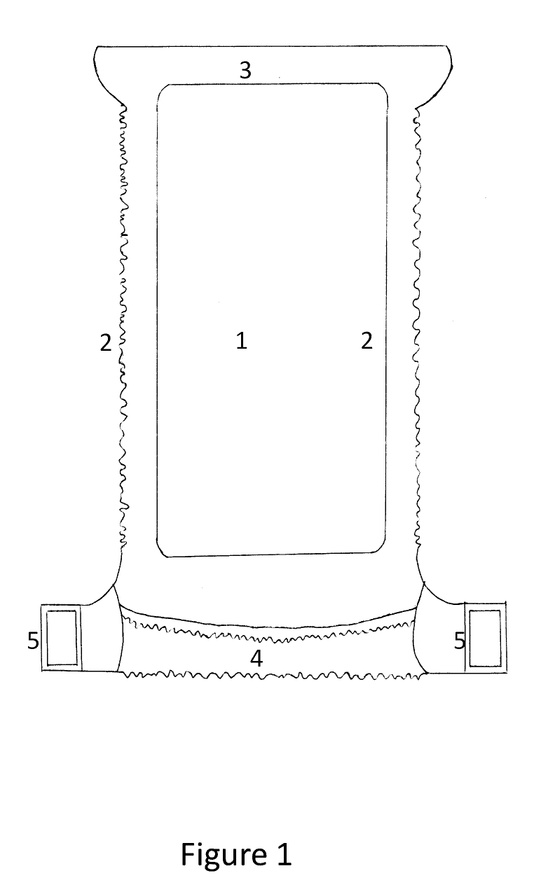

FIG. 2: is a front perspective view of the absorbent article illustrating the operation of the vertical tape urine detection (6).

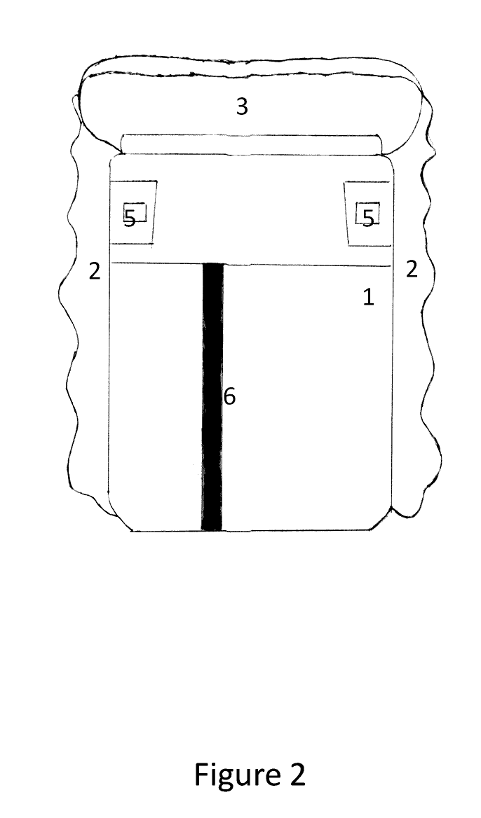

FIG. 3: is a front perspective view of the absorbent article illustrating the operation of the vertical tape urine detection (6) and the vertical tape detection stool (7).

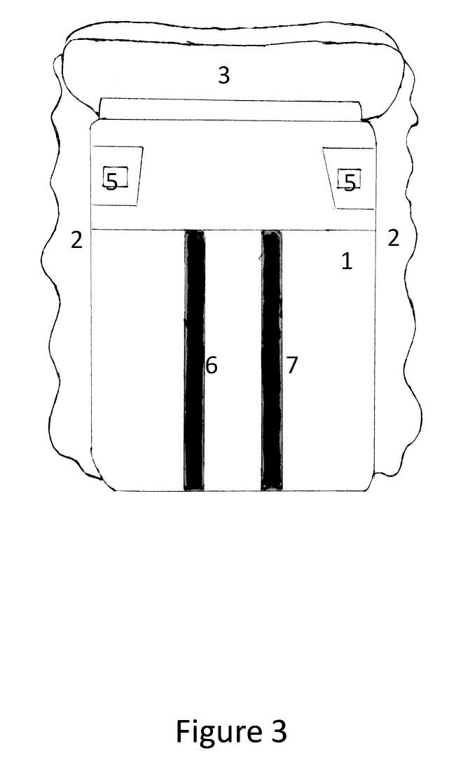

FIG. 4: shows a cross section of how to apply the tapes and how they must have the grooves (8) as a window to allow the small step stool and urine reaching tapes (6) and (7).

1. Disposable absorbent article comprising an absorbent core (1), lateral spring legs (2), front side (3), spring back (4), and tape (5) at the ends of spring back, characterized by a pair of belts on the outside of the absorbent core where a tape (6) has an indicator for detecting urine (6) at pH between 3 and 5 and the other tape (7) has said indicator in an acid solution to detect stool characterized by distinct identification of urine and feces.

2. Disposable Absorbent Article according to claim 1, wherein the ribbons are in a groove (8) of the absorbent core.

3. Disposable absorbent article according to the preceding claims, wherein the ends of the straps are attached to the front side (3) and spring back (4).

4. Disposable absorbent article, wherein the indicator is prepared bromophenol blue 0.1% in 20% ethyl alcohol.

5. Disposable absorbent article, wherein the solution is a mixture of acetic acid with sodium acetate salt.

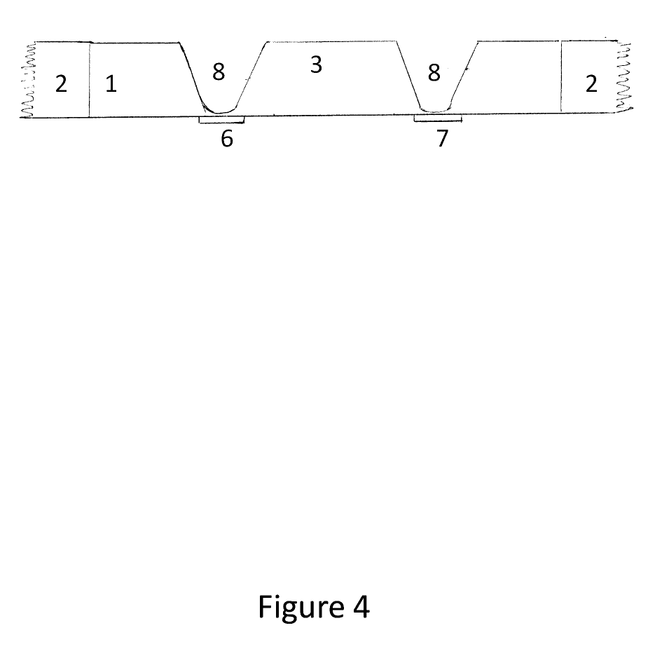

6. Disposable absorbent article, in the process for inclusion of indicator tapes in an absorbent article comprising: the second phase is the formation of the core, which package once the desired shape, is transported by a continuous sheet of paper, which can be on top or around the package. The package is compressed through trouser roller, and is then cut into individual parts. And the third phase of the Armed in which a strip of polyethylene or of an impermeable film fed into the bottom of the package, the headband was previously added to the film by using an applicator so that these materials can join is hot melt adhesive called hot mixture, the elastic are added into this section and glued with adhesive; characterized by a change in the process of inclusion of the indicator tapes as follows: In the second phase, when compressing the package, we need to include in the forming mold two rows (8) by way of windows that allow the small stool and urine, the reaction step may be generated conspicuously and contact the test strips. The grooves must have an entry between 2.0 and 3.0 mm and an output of 1.0 mm, as shown in FIG. 4. In the third phase, the polyethylene strip and including test strips are described above, in the same position the absorbent article which are grooves (8), the tapes will allow detection of fluids also serve as extra support to the strip impermeable polyethylene, in order to avoid any leakage.
